(19) United States
(12) Patent Application Publication
(10) Pub. No.: US 2025/0252590 A1
(43) Pub. Date: Aug. 7, 2025

(54) VOLUMETRIC SHOE SIZING

(71) Applicant: Feel the World, Inc. D/B/A Xero Shoes, Broomfield, CO (US)

(72) Inventor: STEVEN SASHEN, Broomfield, CO (US)

(73) Assignee: Feel the World, Inc. D/B/A Xero Shoes, Broomfield, CO (US)

(21) Appl. No.: 18/855,192

(22) PCT Filed: Apr. 17, 2023
(86) PCT No.: PCT/US2023/018827

§ 371 Date: Oct. 8, 2024

Related U.S. Application Data
(60) Provisional application No. 63/363,134, filed on Apr. 18, 2022.

(51) Int. Cl. G06T 7/62; G06T 7/50; G06T 15/08; G06T 19/20

(52) U.S. Cl. CPC G06T 7/62; G06T 7/50; G06T 15/08; G06T 19/20; G06T 2200/24; G06T 2207/30196; G06T 2219/2016

(57) ABSTRACT

Techniques for automated shoe fitting are disclosed. The present disclosure includes a method including receiving foot data including information about a foot of a user (e.g., user preferences, images, other information), generating a foot model of the foot based on the foot data, where the foot model includes a three-dimensional model, comparing the foot model to a shoe model, where the shoe model includes a three-dimensional model, and displaying individualized shoe information to the user based on the comparison for the user to choose an effective shoe fit. In some examples, the present disclosure describes determining three-dimensional characteristics of a shoe (e.g., via an interior scan), determining shoe data and characteristics (e.g., material characteristics), generating a shoe model, determining parameter ranges for the shoe, and rendering the shoe model. The disclosed technology can prioritize user agency and/or can generate recommendations.

### CROSS-REFERENCE TO RELATED APPLICATION

This application claims the benefit of priority pursuant to 35 U.S.C. § 119 (e) of the Applicant's U.S. provisional patent application No. 63/363,134, filed Apr. 18, 2022, titled “Volumetric Shoe Sizing,” which is hereby incorporated by reference in its entirety for all purposes.

### TECHNICAL FIELD

The present disclosure relates generally to methods to generate shoe fit determinations and recommendations.

### BACKGROUND

People sometimes shop for shoes depending on their needs as a consumer. For example, people may buy shoes for running, hiking, walking, or the like. In some other examples, people may have particular shoe preferences, or may have certain characteristics of their feet that shoes may not be able to accommodate.

Increasingly, people are using ecommerce to shop, rather than shopping at traditional brick and mortar stores. As such, some consumers are using ecommerce to shop for shoes but are unable to physically try the shoes on in person before purchasing the shoes. Shoes and the fit of a shoe (e.g., comfort experienced by the user while wearing the shoe) may be variable based on different brands, types, materials, or the like, and consumers may thus have a difficult time finding the right “fit” online, even shopping for brands with which the customer is familiar. This can result in consumers becoming frustrated with the process, having to return many pairs of shoes or settling for a fit that is not comfortable or otherwise satisfies the consumer's standards. Additionally, retailers, such as shoe stores or shoe manufacturers, may have unhappy customers based on the lack of good fit, even though most of the time this is a consumer selection issue, rather than a real problem with the shoes. The foregoing problems and other problems are caused by deficiencies in current technologies for helping consumers assess shoe sizing, fit, and other characteristics, such as when purchasing shoes using ecommerce.

Existing technologies may provide limited functionality for footwear sizing applications (e.g., applications on a website, on a phone). Some footwear sizing applications utilize photographs of a user's foot to help the user choose a shoe size. However, such applications may operate using insufficient data, insufficient techniques, or both, and consequently provide poor shoe recommendations to the user. Often these applications base a recommendation solely on calculated length and width measurements of the user's foot which, while providing some information to the consumer regarding fit, is not sufficient to address the variability in different styles, manufacturers, materials, and so on.

### SUMMARY

Techniques for volumetric shoe fitting are described. Some embodiments of the present disclosure include a method. In some examples, the method includes receiving foot data including information about a foot of a user, generating a foot model (e.g., three-dimensional) of the foot based on the foot data, comparing the foot model to a shoe model, where the shoe model includes a three-dimensional model, and displaying individualized shoe information to the user based on the comparison.

In some instances, the individualized shoe information may include shape and/or fit characteristics for the shoe, based on material characteristics for the shoe.

Some other embodiments include an apparatus for generating individualized shoe information for a user. In some examples, the apparatus includes a camera configured to take one or more pictures of a foot of the user, a user input interface configured to receive foot data including information about the foot and/or the user, a processing element, and a display. In some examples, the processing element is configured to generate a first model of a foot of the user based on the multiple images, the data, or a combination of these, and compare the first model to a second model of the shoe. In some examples, the first model and the second model are three-dimensional models. In some examples, the display is configured to display individualized shoe information (e.g., fit information) to the user based at least in part on the comparison.

### BRIEF DESCRIPTION OF THE DRAWINGS

FIG. 1 is a diagram of a system for foot model generation.

FIG. 2 is a diagram of a system for shoe scanning and model generation.

FIG. 3 is a flow diagram illustrating a method to generate shoe fit options based on a foot model.

FIG. 4 is a flow diagram illustrating a method for generating and rendering a foot model.

FIG. 5 is a flow diagram illustrating a method for generating and rendering a shoe model.

FIG. 6 is a simplified block diagram of select components of the system of FIG. 1.

FIG. 7 is a diagram illustrating a shoe model and a foot model as displayed on a user interface.

FIG. 8 is a diagram illustrating a shoe model and a foot model as displayed on a user interface.

### SPECIFICATION

The present disclosure is related generally to systems and related methods for identifying shoe fit options for one or more shoes for a user (“system” or “shoe fit system”). In one example, images (e.g., pictures) or other foot information for one or more feet of a user are captured and the system presents a user with a shoe options based on identified foot characteristics and shoe characteristics, where the options may take into account multiple characteristics of both the foot and the shoe. As one example, a user foot model is generated (e.g., by capturing user information about his or her foot) and used along with a shoe model (which may include specific shoe characteristics, such as material deformation data, internal shoe volume, etc.) to generate a fit display and/or information and optionally generate recommendations to the user regarding fit for the shoe. In some instances, the system may generate overlaid or composite displays combining features of the foot model with the shoe model to allow a user to easily visualize how the shoe may fit and feel when worn. For example, a user interface may be displayed to the user that includes a visual overlay of the user's foot within an identified shoe and shoe size such that a user can be presented visual information regarding the expected fit of his or her feet within the shoe selection. This display can allow users to more accurately identify shoe types and sizes that will provide a comfortable and desirable fit. In other examples, the system may also be configured to automatically analyze the user's foot and recommend or select a particular shoe (e.g., shoe model and size).

In some examples, a user may use a program or application provided by the system on a device to determine a shoe selection or identify multiple shoe options based on user information. Such user information may include images captured of one or more feet or other foot data, user preferences (e.g., shoe fit preferences, shoe type preferences, etc.), and/or other user information, such as input information regarding anatomical characteristics. A foot model corresponding to the user (e.g., an individual representation of a foot of the user) is generated based on the user information. The foot model (e.g., user foot model) may include a three-dimensional (3D) model of one or more feet, which may be generated using the foot information (e.g., captured images and/or user information). User information may include any information about the user (e.g., dimensions such as width and length dimensions, the captured images, other images, image extracted from or based on the captured or other images, or any other data about the user's feet), or preferences of the user. The foot model may include multiple models, where the models include a foot or portions thereof (e.g., an arch, a toe, a heel, or the like). The foot model may additionally or alternatively include foot model metadata, measurements (e.g., calculated volume, area, one-dimensional measurements), preferences, other user information, or the like.

The foot model may be analyzed or compared to a shoe model and based on the comparison, one or more shoe options (e.g., shoe size options, shoe model options, shoe material options, or the like) are presented to the user, e.g., via a display of a user device. For example, a user interface can display an overlay or composite view that shows the user's specific foot model within the selected shoe. In one example, characteristics (e.g., features) of the foot model are compared to characteristics of the shoe model and a particular shoe option may be selected if one or more criteria are met (e.g., user preferences, fit parameters, or the like). In some cases, a shoe model combined with the foot model (e.g., superimposed on the foot model) may be rendered and display to the user, e.g., via a display on the user device. For example, the foot model may be displayed within the shoe model (e.g., positioned within the shoe model) so that the user can inspect various portions of the foot and/or shoe models or the combination model, e.g., allow a user to rotate the shoe and foot to see tolerances and fit along multiple angles. For example, the combination model or composite model may be a full 3D visualization and be viewable from multiple angles and directions, e.g., configured to allow 360-degree rotation along multiple axes within the user interface. In some implementations, the foot model and/or the shoe model can be displayed for analysis on the same device used to capture data for the foot model and/or the shoe model, and in some implementations the foot model and/or the shoe model can be displayed on a different device, such as via a website, an application, or the like. For example, information for the foot model and can be captured in-store on a store device and the user can access the foot model and use it to display with information regarding the shoe model on a user device, e.g., a home computer or the like.

As one example, the shoe model may have a varied appearance, such as semitransparent or translucent portions, to allow a user to view the foot model within the shoe model. Specifically, as the shoe model may be representative of a shoe on the foot of the foot model, which may enclose portions of the foot model, by including semitransparent or translucent portions, which may be selectively activated, the user can view the internal fit of the foot model within the shoe model. In some instances, multiple shoe options are determined and displayed as likely best fits for the user. Shoe options may include same shoes of different sizing or different characteristics, or different shoes, different brands, or any combination of these. In some embodiments, in addition to or separate from the shoe options, the system may display numerical or other fit related data. For example, the system may display a distance value (e.g., centimeters or millimeters) representative of the distance from a front wall of the shoe and the user's longest toe. As another example, the system may display a value, such as a force value, that may indicate the expected force or pressure exerted by the shoe on the top or other areas of the user's foot. As yet another example, the system may display volumetric data for the user, e.g., foot volume information that can be used to compare against “internal volume” numbers for shoes.

The system may generate a recommendation and/or options for a user or in some instances may simply indicate the particular size or shoe dimensions for a particular overlay relationship. For example, the system may display the foot model with a first shoe model option and a second shoe model option to allow the user to select which one he or she wishes to purchase. In these examples, the retailer or shoe seller may reduce customer complaints from users as any issues with the shoe selection may be based on the user's choice of a particular shoe model rather than an automated recommendation. Customer complaints on poor fit may often be directed improperly at the retailer due to the customer feeling “duped” into selecting a particular size, such as via a sizing chart, and providing some agency to the customer via the various overlay options may help to reduce customer complaints. In these instances, the system may automatically determine options or shoe models to display to the user, but the actual selection for a particular shoe, such as to purchase a shoe in a particular size, may be left to the user. In other implementations, however, the system may automatically generate the selections on shoe size and selection based on the foot model and shoe model information and comparison.

In some embodiments, the foot model may be used to provide global recommendations or information to a user regarding multiple types of shoes, e.g., global shoe sizing information. In these embodiments, the system can generate output to the user that may be applicable to many different types of shoes. Alternatively or additionally, the system may also provide specific fit information to user a regarding a selected shoe or set of shoes. In these examples, the system may indicate whether the user's selected shoe and size may fit the user's foot and/or may include information regarding the fit of the selected shoe on the user's foot, e.g., this shoe will have a tight fit and be a bit snug at the top left surface of the right and left feet. This information may be based on threshold distances/spacing at different locations between the foot model and the shoe model and/or the shoe characteristics.

In other embodiments, the foot model may be used to filter or otherwise select shoe options to present to a user. For example, at an ecommerce site, a user can upload foot information that can be used to generate the foot model (or the user or application may directly upload the foot model to the server for the site or a related site). Using the foot model, the ecommerce server may then only serve up options to a user that are likely to fit, e.g., within a predetermined threshold range for various parameters, such as length, spacing, etc. In this manner, the user can easily view large categories of shoes without being overwhelmed by the number of options, e.g., only running shoes that are likely to fit the user are presented rather than all running shoes in that user's length (shoe size).

In some other examples a shoe model is generated based on shoe characteristics, which may be determined from scanning technology to determine dimensions of various portions of the shoe (e.g., an inside of the shoe), shoe information (e.g., design drawings), one or more images captured of the shoe, or a combination of the same. The shoe model may include one or more three-dimensional models of one or more shoes, which may be constructed using the images, data from the scanning technology, the shoe information, a model of the shoe (e.g., a computer-aided design (CAD) model), or the like, or a combination of these. In some examples, the shoe model may include one or more portions of a shoe (e.g., an arch area, a toe area, a heel area, or the like). The shoe model may additionally or alternatively include shoe model metadata, measurements (e.g., calculated volume, area, one-dimensional measurements), shoe material, other shoe information such as characteristics related to performance of the shoe, etc.

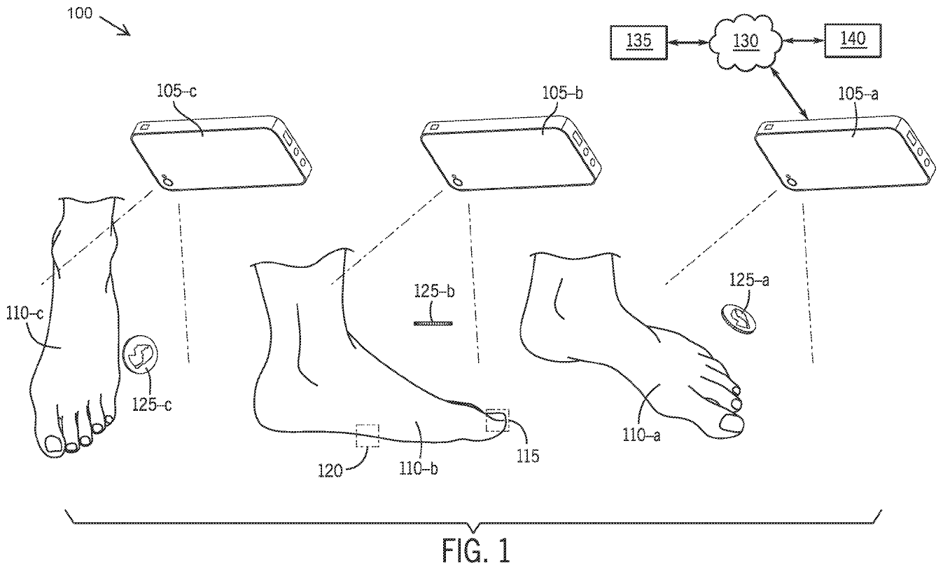

FIG. 1 illustrates a foot model generation system 100. As shown in FIG. 1, the foot model generation system 100 includes devices 105-a, 105-b, and 105-c, foot 110-a, foot 110-b, foot 110-c, first point of interest 115, second point of interest 120, and reference items 125-a, 125-b, and 125-c. Devices 105 may include a display, a processing element, an input and/or output, memory, one or more sensors, a power source, or any combination of these, and devices 105, e.g., may be a smartphone, wearable device, tablet computer, or the like. In many instances, the devices 105 may correspond to a user device. Foot model generation system 100 may capture data from a user's foot, e.g., one or more pictures of a foot 110 (e.g., from various angles, such as those depicted by foot 110-a, foot 110-b, and foot 110-c) (e.g., via a device 105). Foot model generation system 100 or shoe fit system may generate a foot model, comparing a foot model and a shoe model, and displaying either or both models, one or more shoe options, or both, by a device 105 (or via an external display, such as on a user's smart phone display, tablet display, and/or a computer display). While in FIG. 1 the foot model generation system 100 is shown with a phone as a device 105, it may be appreciated that any device may be used as a device 105, and in some cases, a camera used for taking images may be separate from a device 105. Further, while the first point of interest 115 and the second point of interest 120 may depict a foot arch and a toe, respectively, these locations are arbitrary and it may be appreciated that any location on any foot 110 may be a point of interest. Further still, while the reference items 125 may depict a coin, it may be appreciated that any item, device, projection, or the like may serve as a reference item 125, e.g., any item with a generally known or detectable size that can be used as a reference for unknown sizes, such as the user's foot. In some implementations, the system 100 can generate a foot model without using a reference item 125. For example, the system 100 may utilizing multiple sensors to detect depth information, without the use of a scaling or reference item and/or capture data from multiple viewpoints that may be useful in recreating the depth or other dimensional information. In these instances, the system 100 may more easily be able to capture the foot information as the user may not need to retrieve or identify a reference item.

In some examples, a device 105 may use images of a foot 110 (e.g., foot 110-a), other information about the foot 110 of the user, or both, to generate a foot model. A device 105 may take one or more images of a foot 110 from various angles, such as those represented by foot 110-a, foot 110-b, and foot 110-c. Such images may include a reference item 125 to provide a reference measurement for other things in the image, e.g., a known length or height that can be used to estimate or determine length and/or height of other elements within the image. In other examples, the foot information may be retrieved or gathered in other manners, e.g., directly capturing depth information, such as via light detection and ranging (LIDAR), depth sensing cameras, laser scanning, white light scanning, computerized tomography (CT) scanning, x-ray scanning, video, augmented reality (AR) scanning techniques, or the like. In these instances, the device may determine volumetric information directly or may capture other information that can be used to calculate volumetric information for the foot of the user. In these and other implementations, the foot information can be used to generate a foot model without using a reference item 125. For example, the user device itself may scan the area and foot information and generate the dimensional data needed to create the foot model.

A device 105 may optionally receive other information about the foot 110 (e.g., user may input such other information to a device 105). Such other information may include user preferences. For example, a user may have a preference for an amount of space desired at the front of the shoe, a material preference, a tightness or snugness preference, a Velcro preference, a laces preference, or any other preference. Additionally or alternatively, such other information may include conditions (e.g., Morton's toe, inserts such as orthotics and related information, scoliosis and related information) known by the user, one or more foot dimensions or classifications (e.g., foot width, length) known by the user, a shoe size known by the user, an environmental condition known by the user (e.g., trail running, street running, track running, biking, walking, hiking, or the like), or any other information about the foot 110 or corresponding user. In other examples, the foot model may be generated without receiving user preference information and the user preference information may be requested or received at a fitting operation (if received at all). For example, one benefit of the system may be to allow recommendations/assessments on shoe fit without the need to receive input from the user in the form of user preferences, especially as users may have a disparity between what they believe to be a good fit and what they actually think is a good fit in reality.

A device 105 may generate a foot model based on the images, the other information, or both. For example, a device 105 may generate one or more three-dimensional models of a foot 110 or portions of a foot 110 (e.g., first point of interest 115, second point of interest 120, such as a big toe, heel, or both). The foot model may additionally or alternatively include the other information as metadata, as a part of the three-dimensional model, or for reference elsewhere (e.g., in other files). The foot model may include determined measurements, calculations, or classifications by a device 105 based on the three-dimensional model, the other information, or both. For example, the foot model may include a calculated volume of a foot 110 (e.g., calculated based on the three-dimensional model), a calculated volume of a portion of a foot 110 (e.g., at first point of interest 115, second point of interest 120, such as a big toe, heel, or both), a calculated area (e.g., a cross-sectional area, a surface area), a calculated length measurement (e.g., one or more widths, lengths, heights at various portions of a foot 110, such as at first point of view 115, second point of view 120, such as a big toe, heel, or both), a calculated angle (e.g., one or more foot arch angles), a determined foot shape (e.g., neutral, straight, curved), a determined toe shape, any other classifications (e.g., pronation, supination, flat foot, low arch, medium arch, high arch), or the like. In some other examples, a device 105 may compare different portions of a foot 110 (e.g., compare volumes, areas, one-dimensional measurements for different parts of a foot 110). For example, a device 105 may compare a height measurement of a foot 110 to a width measurement of a foot 110, or may compare a volume measurement of a foot 110 to the width measurement, as a high volume foot inside a wide shoe may feel narrow to a user. The foot model may include results of such comparisons.

A device 105 (e.g., computing device and/or server) may compare the foot model to a shoe model to determine a selection of shoes to display to the user. For example, a device 105 may map the volume of a foot 110 to an internal volume of a shoe. A device 105 may compare volumes of portions of a foot 110 with corresponding portions of a shoe. A device 105 may additionally or alternatively compare two-dimensional (area, cross section, etc.) and one-dimensional (length, width, height) measurements of a foot 110 with corresponding portions of a shoe. In some cases, a fit may be good if a difference of such volume, area, or distance measurements of a foot 110 and a shoe are within a threshold (e.g., snugness). A good fit evaluation may change based on any user preferences or other user information as discussed previously. For example, a user may request a tight fit at a front of a shoe, and thus a threshold difference in volumes at the front of a foot/shoe may decrease.

A device 105 may map the volume of a foot 110 to an internal volume of a shoe. However, the volume of the shoe may change or have variability due to factors including a material of the shoe, material thickness, material elasticity or stretch, whether or not the material is scored, shoe environment (e.g., walking, running), shape of shoe and shoe bed, wideness of the shoe relative to shoe volume (e.g., a high volume foot inside a wide shoe may feel narrow to a user), such as at certain locations within the shoe, compressibility or compression rate of shoe materials, shape and thickness of an insole or orthotic at various portions, shape and thickness of a sole of the shoe, effects of tightening laces, Velcro or other hook and loop fasteners, two-way stretch effects on material, four-way stretch effects on material, fabric biases, temperature considerations, or the like. Additionally, the foregoing factors and other factors may change over time, such as based on an amount of use of a shoe or ordinary wear of the shoe over time. As such, a device 105 may account for such changes in volume in addition to user preferences and volume and/or fit comparisons between a foot 110 and a shoe. For example, a device 105 may calculate one or more volume ranges at various locations on the shoe (e.g., first point of interest 115, second point of interest 120) to determine a good fit. Based on such factors, preferences, volume ranges, and the like, a device 105, an internal measuring device, or any combination of these, may calculate a total maximum volume, minimum volume, or both, for a shoe.

A device 105 may compare a foot 110 volume (or a volume of a portion of foot 110) to shoe option volumes based on such factors and user preferences, and display such shoe options to the user. For example, device 105 may determine one or more locations or areas of interference or minimal spacing where a foot 110 may interfere with a shoe (e.g., when comparing or mapping the volumes or models of the foot and shoe to each other). For example, device 105 may determine such locations based on volume variability at various portions of a shoe according to the factors, preferences, volume ranges, or the like as described previously. For example, device 105 may determine that material stretch at a toe portion of the shoe is relatively negligible or small, and may determine that a user's foot 110 may interfere or have minimal spacing at the toe portion of the shoe.

As one example, select points of the shoe may be used as representative locations for comparing the fit as compared to the foot model. In other words, rather than using an entire volumetric comparison which may identify all possible interferences or spacing issues between a particular shoe and a particular foot, to expedite the analysis and the burden on the computation, the comparison may be done at discrete locations. These locations may be selected by the user or may be default options.

A device 105 may select one or more shoes to display to the user that provide a desired volumetric fit based on user information and user fit preferences, e.g., a fit within a select tolerance range, which may vary at different locations for the foot. For example, a tighter tolerance or distance between the edge of the user's heel and the back wall of the shoe may be satisfactory for a match as compared to a larger distance between the edge of a user's longest toe and the front wall of the shoe. In some cases, the display may include a rendering of both the shoe model and the foot model together, such as to generate a composite or overlay view. For example, the rendering may include the foot model inside the shoe model. In some cases, the shoe model, foot model, or both may be slightly transparent, completely transparent, or opaque for a user to investigate the fit of the shoe on a foot 110. In some cases, the display may include multiple renderings displaying multiple views of the shoe model and the foot model. The overlay or composite view allows a user to visualize the shoe fit, which may be more similar to physically trying the shoe on, allowing a better shoe selection for the user than typical ecommerce sizing options. Device 105 may display one or more shoe models of a given shoe (e.g., according to a maximum volume of the shoe or portions thereof, a minimum volume of the shoe or portions thereof, or the like). In some cases, device 105 may display one or more shoes from one or more manufacturers (e.g., device 105 may display results from an ecommerce site, which may include one or more brands, manufacturers or the like). In some cases, device 105 may display the one or more shoes in an AR setting or a virtual reality (VR) setting, and the user may be able to “try on” shoes in AR or VR, or view shoes in AR or VR alongside a user foot 110.

In some examples, a device 105 (e.g., device 105-a) may communicate with a first server 130 (e.g., a cloud server). For example, device 105-a may transmit images of a foot 110 to the first server 130. Additionally or alternatively, device 105-a may transmit other user information, preferences, or the like. First server 130 may transmit the received images, other user information, preferences, or the like, to a second server 135 (e.g., a processing server, a model server, or the like), to a database 140 (e.g., a characteristics database), or both. Database 140 may store the received images, other user information, preferences, or the like. Second server 135 may compute, or process, or the like, the received images, other user information, preferences, or a combination of these. That is, a device 105 may transmit information to second server 135 for processing, as such processing may be resource intensive and device 105 may not have sufficient computing power, or be able to process the information in a timely manner. In some examples, device 105 may perform some aspects of processing of the images, other user information, preferences, or a combination of these, and transmit the processed information, and additionally or alternatively transmit all or some portions of the original images, user information, preferences, to the second server 135 (e.g., via first server 130) for further processing. In some examples, first server 130 (e.g., a cloud server) may perform some or all processing on the received information from the device 105 (e.g., and may transmit to second server 135 for further processing). As such, any calculations, processing, or the like described herein as being performed by device 105 or device 205 may be performed by first server 130, second server 135, or both, or a combination of such devices and servers.

A device 105 may receive the processed or stored information from first server 130 (e.g., a cloud server). First server 130 may receive information from second server 135, database 140, or both. For example, first server 130 may receive processed information from second server 135, may receive stored information from database 140, or both. The processed information received by a device 105 may include one or more foot models, which may include various calculations, measurements, 3D models, shoe models, the original images, the original user information, preferences, or a combination of these.

In some implementations, the device 105 generates the foot model using data (e.g., images and/or other foot information) received from a different device, such as a specialized scanning device for capturing data related to a foot. For example, a specialized scanning device may be utilized to capture data within a store location and the foot model may be stored in a database and accessible to a user on a separate user device, e.g., a home computer. In this manner, the user may go into a physical store to have his or her foot scanned and then may utilize the foot model generated as part of the scanning process to assist in selecting shoes purchased from an ecommerce platform or otherwise available on-line.

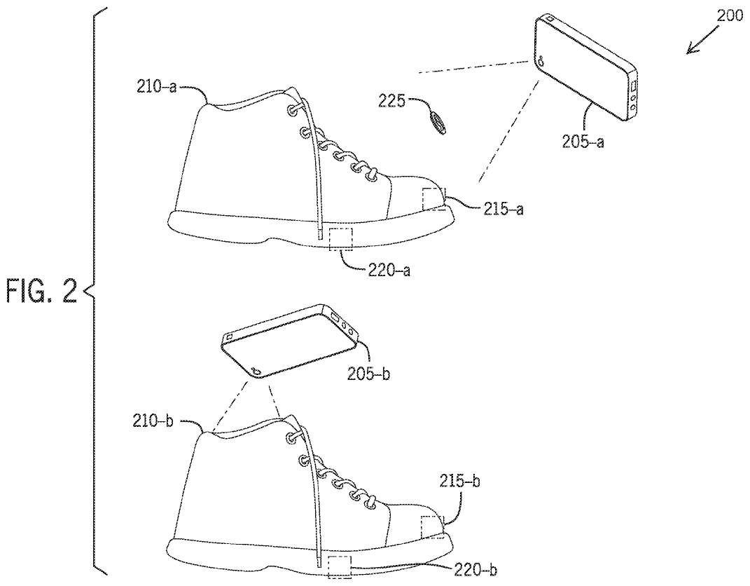

FIG. 2 illustrates a shoe scanning and model generation system 200. As shown in FIG. 2, the shoe scanning and model generation system 200 includes a device 205-a, device 205-b, shoe 210-a, shoe 210-b, first point of interest 215-a and 215-b, second point of view 220-a and 220-b, and reference item 225. Device 205 may include a display, a processing element, an input and/or output, memory, one or more sensors, a power source, or any combination of these. Shoe scanning and model generation system 200 may depict device 205 taking one or more images of a shoe 210 (e.g., from various angles). Shoe scanning and model generation system 200 may additionally or alternatively depict processing and generation of a shoe model, comparing a shoe model and a foot model, and displaying either or both models, one or more shoe options, or both, by the device 205 (or via an external display). While the shoe scanning and model generation system 200 may depict a phone as device 205, it may be appreciated that any device may be used as device 205, and in some cases, a camera used for taking images may be separate from device 205. In particular, in some examples, device 205-b may comprise a laser device for measuring an internal volume of shoe 210-b. In some cases. Device 205-b may use LIDAR technology and/or other technologies for measuring the internal volume of shoe 210-b. The internal volume of shoe 210-b may additionally or alternatively be measured via an inflatable device (e.g., an inflatable device such as a balloon, foam, or the like, with pressure sensors at various locations of the shoe). In these and other implementations, various measurements can be taken of the interior of the shoe 210-b, such as volume of one or more portions of the interior of the shoe 210-b, area, cross section, length, width, pressure or force at various points, and so forth. Further, while the first point of interest 215 and the second point of interest 220 may depict a shoe sole and a toe area, respectively, these locations are arbitrary and it may be appreciated that any location on any shoe 210 may be a point of interest. Further still, while the reference item 225 may depict a coin, it may be appreciated that any item, device, projection, or the like may serve as reference item 225. In some implementations, the system 200 can generate a shoe model without using a reference item 125.

To generate a shoe model, a device 205 may take one or more images of a shoe 210, scan an inside of a shoe 210 (e.g., via a laser device), utilize one or more measuring devices (e.g., force, pressure, or strain gauges), and/or use other shoe information (e.g., stored at a database, design drawings). The shoe model may include or be based on internal shoe volume information. For example, the shoe model may include internal shoe volume information for one or more portions of the inside of a shoe 210 (e.g., a toe portion, a heel portion, or any other portion or combination of portions). Device 205-a may generate a three-dimensional model based on taking the one or more images of a shoe 210 and/or various measurements of the interior of the shoe 210. In some other examples, device 205-b may be a laser device and may perform a 3D scan of the inside of shoe 210-b to determine an inside volume of shoe 210-b. Device 205-b may determine a volume range of shoe 210-b by considering compression of one or more materials (e.g., via data from the one or more force gauges) (e.g., at first point of interest 215, second point of interest 220). For example, the internal volume of shoe 210-b may be different depending on how much a shoe material is being compressed, among other factors. The internal volume of shoe 210-b may change based on factors such as material thickness, material elasticity or stretch, whether or not the material is scored, shoe environment (e.g., walking, running), shape of shoe and shoe bed, wideness of the shoe relative to shoe volume (e.g., a high volume foot inside a wide shoe may feel narrow to a user), compressibility or compression rate of shoe materials, shape and thickness of an insole or orthotic at various portions, shape and thickness of a sole of the shoe, effects of tightening laces, Velcro or other hook and loop fasteners, two way stretch effects on material, four way stretch effects on material, fabric biases, temperature considerations, or the like. Additionally, the foregoing factors and other factors may change over time, such as based on an amount of use of a shoe or ordinary wear of the shoe over time. Device 205-b may include one or more three-dimensional models (e.g., an inside, outside of a shoe 210) in a shoe model, and additionally or alternatively other information such as material, shoe shape, or any of the other factors described previously. A device 205 may send the shoe model to another device for comparison to a foot model.

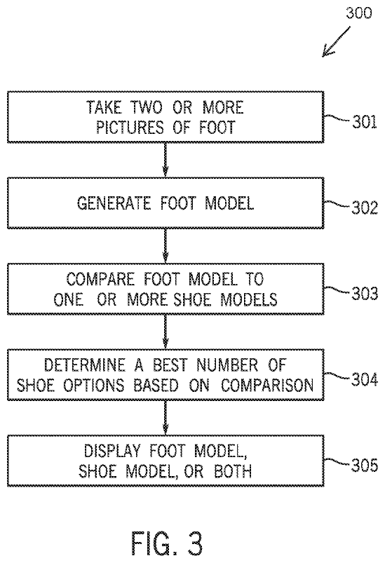

FIG. 3 illustrates a flow diagram 300 for a method of generating a foot model for comparison to a shoe model, and displaying one or both of the models. The method begins with operation 301, taking two or more images of a foot. For example, images of a foot may be taken from various angles by a camera. In other examples, foot information may be retrieved or gathered in other manners, e.g., directly capturing depth information, such as via LIDAR, depth sensing cameras, or the like, which can be used in addition to images and/or as an alternative to images. In these instances, the device may determine volumetric information directly or may capture other information that can be used to calculate volumetric information for the foot of the user. This may be one example of receiving foot data including information about a foot of a user, as the images may be included in the foot data. Other foot data may include preferences, other information, or the like. It should be noted that the foot data may include images captured from two or more viewpoints, e.g., at different locations relative to the foot in order to generate the desired dimensional (e.g., length, width, and/or depth) information without the use of or assisted by a reference item.

After operation 301, the method proceeds to operation 302, generating a foot model. This may be one example of generating a foot model of the foot based on the foot data, where the foot model includes a three-dimensional model. The foot model may be based on a combination of some or all information related to a user foot, such as the images taken or received at operation 301, user preferences, or other information.

After operation 302, the method proceeds to operation 303, comparing a foot model to one or more shoe models. This may be one example of comparing the foot model to a shoe model, where the shoe model includes a three-dimensional model. In some examples, the shoe model for comparison may be a selected shoe (e.g., predetermined shoe) (e.g., by the user), or multiple shoes (e.g., different shoe models, such as different brands of a same shoe type (e.g., one or more running shoes or any selection of shoes identified by the user)) (e.g., multiple shoes from a database of shoes). When comparing the foot model to multiple shoes, the method may include a filtering or thresholding algorithm or method that eliminates one or more shoes from the comparison (eliminating for output/display to the user) based on one or more interferences (e.g., in preferences, in physical model (e.g., volumetric) conflicts based on the comparison of the foot and shoe models). For example, the method may filter out shoes with parameters or measurements that are within a range (e.g., user preference, foot does not fit within the interior of the shoe or does not fit within portions of the interior of the shoe). The method may keep shoes that have parameters or measurements that are within a range (e.g., user preference, foot fits within the interior of the shoe or fits within portions of the interior of the shoe).

Comparing the models may involve comparing foot volume measurements, area measurements, length/width/height measurements, or a combination of these, to corresponding portions of a shoe. For example, a volume of a foot may be compared to different volume ranges of an internal volume of a shoe (e.g., due to material compression, or other factors as described previously), and thus a good fit according to user preferences may be determined. As one example, distances or tolerances between the outer surface of the foot model and the interior surfaces of the shoe at different locations may be determined. The system may analyze a few points, such as common locations for “fit” feel in a physical shoe, such as the space between the big toe and front wall of the shoe, space between the top of the foot and the tongue of the shoe, space between sides of the shoe and sides of the user's foot, and/or space between the heel and back wall of the shoe. In some instances, the tolerances may be acceptable in different thresholds for different locations on the shoe/foot. In some instances, fewer locations are analyzed and in other embodiments, more locations are analyzed. In some instances, the comparison of the foot model to the one or more shoe models can take into account properties of materials included in the one or more shoes, such as texture, flexibility, rigidity, hardness, softness, and so forth. For example, determining distances or tolerances between a foot model and a shoe model can include determining whether at least a portion of a shoe is flexible, such that the distance or tolerance can change when the shoe flexes (e.g., in response to being worn or used).

After operation 303, the method proceeds to operation 304, determining a selection of shoe options based on the comparison. Any number of shoe models are compared to the foot model, and a number of acceptable shoes options are selected based on repeated comparisons from operation 303. For example, shoes that would not satisfy minimum spacing thresholds for certain foot locations may be not considered in the selection, whereas shoes that satisfy or meet the spacing thresholds or other user preferences may be included. In some cases, the method may include determining one or more shoes from one or more manufacturers or types (e.g., determine results from an ecommerce site based on user preferences, which may include one or more brands, manufacturers or the like).

After operation 304, the method proceeds to operation 305, displaying the foot model, shoe model, or both. This may be one example of displaying individualized shoe information to the user based on the comparison. The foot model, shoe model, or both, may be displayed to the user. In some examples, volumetric information of the foot model, the shoe model, or both, may be displayed in addition, or alternatively, or as a part of the foot model, the shoe model, or both. In some examples, the foot model is displayed as inside the shoe model. In some examples, the shoe model is superimposed or overlaid on the foot model. In some examples, the foot model, shoe model, or both are completely transparent, somewhat transparent, or opaque. The user may be able to investigate one or both models for one or more shoe options to determine a preferred shoe. The user may be able to activate or deactivate the transparency of the shoe model or foot model representation while also spinning or otherwise rotating the combined model or representation. In this manner, the user can analyze the fit at multiple angles and orientations and for multiple different parts of the shoe and the foot. The user may be able to select a length or any other dimension of a shoe based on preference, which may change the volumetric information of a model (e.g., the shoe model) and the associated display. In some cases, the method may include displaying one or more shoes from one or more manufacturers or types (e.g., display results from an ecommerce site based on user preferences, which may include one or more brands, manufacturers or the like). This allows a holistic and comprehensive analysis on how a particular shoe may fit on his or her foot. Advantageously, the disclosed technology allows the user to select appropriate shoes based on the user's preferences, informed by the holistic and comprehensive analysis, thereby increasing the likelihood that the fit will be consistent with the user's expectations and preferences.

In some implementations, the method includes displaying and/or providing other information, such as a recommended size, an evaluation of the fit of the shoe represented by the shoe model, and/or other information about the shoe model or the foot model. The user can use the displayed information to make a selection of a shoe, a size of a shoe, or the like, and the user can initiate a purchase of a selected shoe (e.g., by placing the selected shoe in a shopping cart).

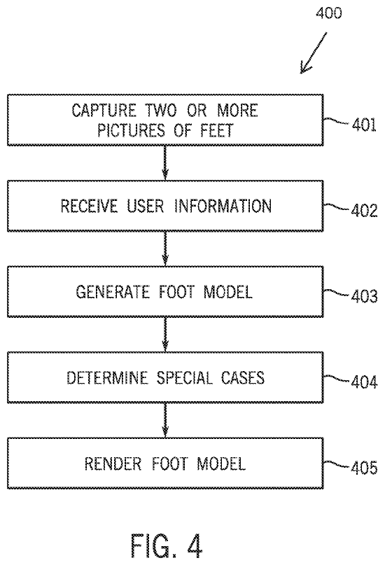

FIG. 4 illustrates a flow diagram 400 for a method of generating and rendering a foot model. The method begins with operation 401, capturing two or more images of feet. This may be one example of receiving foot data including information about a foot of a user, as the images may be included in the foot data.

After operation 401, the method proceeds to operation 402, receiving user information. This may be one example of receiving foot data including information about a foot of a user. Such user information may include user preferences or other user information, such as various conditions (e.g., Morton's toe, inserts such as orthotics and related information, scoliosis and related information) known by the user, one or more foot dimensions or classifications (e.g., foot width, length) known by the user, a shoe size known by the user, an environmental condition known by the user (e.g., trail running, street running, track running, biking, walking, hiking, or the like), or any other information about the foot 110 or corresponding user. In some examples, one or more of such user information may be automatically identified by image capturing or any of the other data capturing techniques described previously (e.g., LIDAR, or the like). In some examples, such user information may be identified by the image capturing techniques, and confirmation of such identified user information (e.g., Morton's toe) may be inputted by the user. For example, a display may display a confirmation message of the identified user information, and the user may confirm or deny the existence of the identified user information.

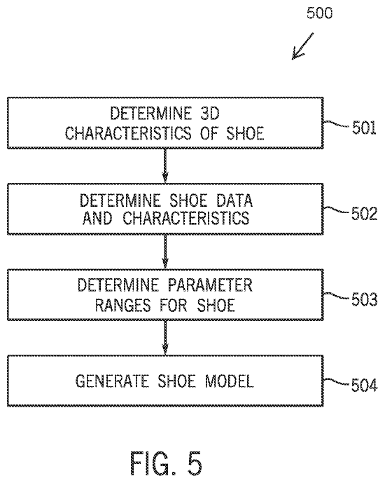

In some implementations, the user information received at operation 402 can include information about a preferred shoe of the user. For example, the user can indicate a type and size of a shoe the user currently wears, which the user believes to be a good fit. Additionally or alternatively, the user can provide or generate a shoe model for the preferred shoe (e.g., using the method illustrated in FIG. 5), which can be used for comparison to other shoe models to determine a good fit for the user. In other words, information about the preferred shoe can be received and used to identify other shoes with a similar fit.

After operation 402, the method proceeds to operation 403, generating a foot model. The foot model may be a three-dimensional shoe model, and may be constructed using the images, the user information, or a combination of these. Any software and/or device(s) may be used for constructing a three-dimensional model from such images and user information.

After operation 403, the method proceeds to operation 404, determining special cases. For example, a user may not indicate some information in the user information, but special cases may be determined by analyzing the foot model. For example, calculated angles and distances of a foot arch may reveal that a user has a flat foot. Any condition or classification may be determined by analyzing one-dimensional, two-dimensional, or three-dimensional measurements of the foot model. Thus, a good fit shoe may be able to be recommended according to such cases. For example, an option may be displayed to the user based on the condition or classification to ask if they would like one or more features in the shoe based on the condition or classification (e.g., may display an option to the user if the user would like built in arch support based on the flat foot determination).

After operation 404, the method proceeds to operation 405, rendering the foot model. The foot model may be displayed on a device display to the user, and the foot model may be able to be inspected by the user at various parts of the foot model. The user information may additionally or alternatively be rendered to the user on the display. In some cases, the foot model may be combined with a shoe model as described with reference to FIG. 3.

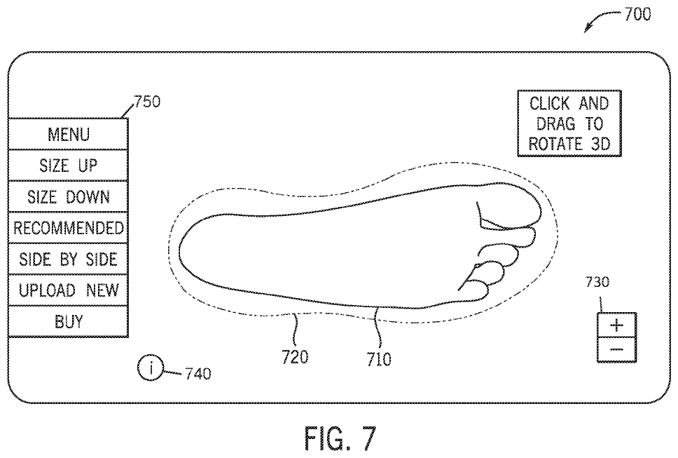

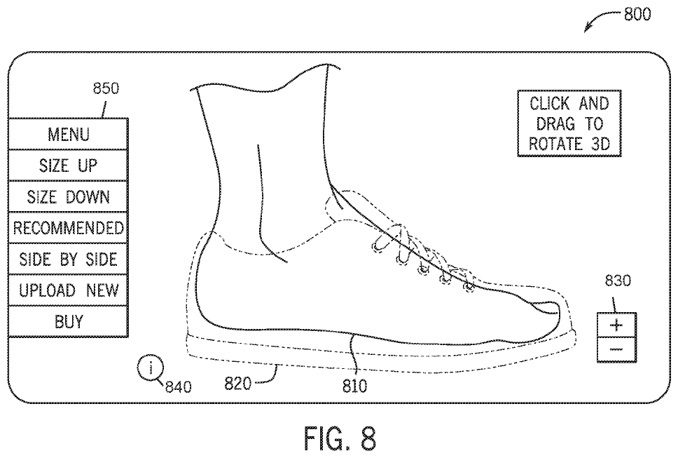

In some implementations, the method includes providing a user interface to display the foot model, such as a user interface as described in FIGS. 7 and 8. For example, the method can include displaying the foot model in the user interface in a combination or composite model or view (e.g., combined or overlaid on a shoe model) to allow a user to evaluate the fit of a particular shoe. The user interface can provide various functions to facilitate evaluation of the fit. For example, the user interface can include a narrative statement regarding the fit based on the information received at operation 402, the foot model rendered at operation 405, and/or the shoe model, such as an evaluation provided by the system regarding whether a shoe represented by a shoe model will be a good fit for a foot represented by the foot model. In some implementations, the user interface allows the user to zoom in or zoom out, such as by selecting one or more sliders or icons or by using a touch screen (e.g., pinching moving two fingers closer together to zoom in or farther apart to zoom out). In some implementations, the user interface allows the user to rotate a view of the model, such as by selecting an icon or slider or by clicking a spot within the user interface and dragging in the direction of rotation.

In some implementations, the user interface allows the user to evaluate the fit of different sizes of a same shoe, such as by selecting an icon for increasing or decreasing size. In response to a selection of the size up or size down icon, the system can update the shoe model to be the next size larger or the next size smaller.

In some implementations, the user interface includes a recommendation icon that, when selected, causes the system to generate one or more recommendations, such as a recommended shoe size for the shoe represented by the shoe model. Additionally or alternatively, the system can recommend a different shoe for the user to evaluate based on the rendered foot model.

In some implementations, the user interface includes an icon to activate a side-by-side view, such as for evaluating multiple shoe models. When activated, the side-by-side view causes display of at least two combination or composite models, each combination or composite model corresponding to a different shoe model overlaid on a same foot model. The side-by-side view can be arranged in various ways, such as in a single row or column or in a grid view comprising multiple rows or columns. The side-by-side view facilitates comparison of the fit of multiple kinds of shoes and/or comparison of different sizes of the same kind of shoe.

In some implementations, the user interface includes an icon to upload new foot images and/or new user information. For example, the user can determine that the foot model rendered at operation 405 needs to be updated (e.g., because it appears to be inaccurate or of insufficient quality), and the user can choose to upload new foot images and/or new user information to update the foot model.

In the foregoing and other implementations, the method illustrated in the flow diagram 400 can include providing the user interface, receiving a user input via the user interface (e.g., to rotate, zoom, change shoe size, generate a recommendation, activate side-by-side view), and updating the display based on the received user input. Updating the display can include changing a viewing angle, presenting a zoomed-in or zoomed-out view proportional to the input, displaying a different shoe model (e.g., a size up or a size down), activating a side-by-side view, and so forth.

FIG. 5 illustrates a flow diagram 500 for a method of generating and rendering a shoe model. The method begins with operation 501, determining 3D characteristics of a shoe. This may include capturing one or more images of a shoe (e.g., exterior of a shoe), performing a 3D scan on the inside of a shoe (e.g., via a laser device), measuring compressibility and other factors of shoe materials with a force gauge or similar device, or any combination of these. For example, the 3D characteristics can be determined by measuring various characteristics of an interior of the shoe, such as an overall volume, a volume of one or more portions, lengths, widths, heights, cross sections, areas, and/or the like.

After operation 501, the method proceeds to operation 502, determining shoe data and characteristics. In some examples, the shoe data and characteristics may include data obtained from operation 501. In some other examples, the shoe data and characteristics may include information obtained from a database or memory. Such shoe data and characteristics include, for example, volume, area, length, width, and height at different portions of the shoe, shoe materials, material widths, presence of laces, and other data and characteristics as described previously.

After operation 502, the method proceeds to operation 503, determining parameter ranges for the shoe. For example, parameter ranges can be determined to indicate a range of movement or deformation of a shoe. In some instances, the parameter ranges can indicate whether the shoe will flex or stretch in one or more areas. For example, although a shoe might have a certain specified width, length, or other dimension, the shoe might be able to flex to fit a foot that is wider, longer, thicker, and so forth when the flexibility of the show is taken into account.

After operation 503, the method proceeds to operation 504, generating the shoe model. The shoe model may be a three-dimensional shoe model, and may be constructed using the images, the shoe data and characteristics, the parameter ranges, or a combination of these. Any software and/or device(s) may be used for constructing a three-dimensional model from such images shoe data and characteristics. The shoe model may be displayed on a device display to the user, and the shoe model may be able to be inspected by the user at various parts of the shoe model. The user information may additionally or alternatively be rendered to the user on the display. In some cases, the shoe model may be combined with a foot model as described with reference to FIG. 3.

The methods illustrated in flow diagrams, 300, 400, and 500, can be performed in any order and/or combined in various ways. Additionally, operations can be added to or removed from any of the depicted methods without deviating from the teachings of the present disclosure, and one or more operations of the depicted methods can be performed in parallel. The depicted methods can be performed, for example, by the shoe fit system, such as the system 100 of FIG. 1 and/or the system 200 of FIG. 2, which can be combined into a single shoe fit system.

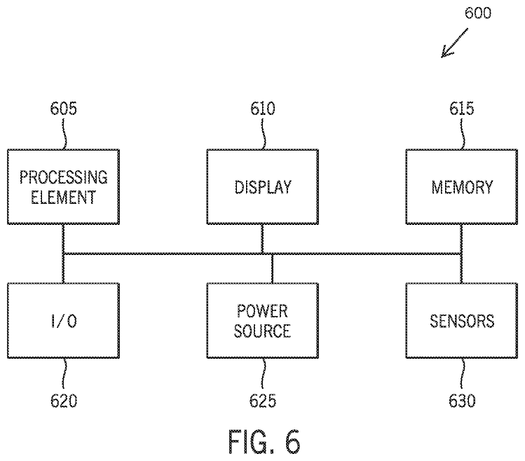

FIG. 6 illustrates an exemplary block diagram of a device 600, for example, a device 105 or a device 205, or both, including computing resources and components. A device 105 or device 205, or both, may include one or more processing elements 605, displays 610, one or more memory 615, an input/output interface 620, power sources 625, and sensors 630, each of which may be in communication either directly or indirectly.

The processing element 605 is any type of electronic device capable of processing, receiving, and/or transmitting instructions. For example, the processing element 605 may be a microprocessor or microcontroller. Additionally, it should be noted that select components of the foot model generation system 100 or shoe scanning and model generation system 200 may be controlled by a first processor and other components may be controlled by a second processor, where the first and second processors may or may not be in communication with each other. The devices 105 and 205 may include one or more processing elements 605 or may utilize processing elements included in other components.

The display 610 provides visual output to a user and optionally may receive user input (e.g., through a touch screen interface). The display 610 may be substantially any type of electronic display, including a liquid crystal display, organic liquid crystal display, and so on. The type and arrangement of the display depends on the desired visual information to be transmitted to the (e.g., can be incorporated into a wearable item such as glasses, or may be a television or large display, or a screen on a mobile device).

The memory 615 stores data used by the device 600 to store instructions for the processing element 605, as well as store positional and content data for the foot model generation system 100 or shoe scanning and model generation system 200. For example, the memory 615 may store data or content, such as feedback reference ranges, values, images, graphics, and the like. The memory 615 may be, for example, magneto-optical storage, read only memory, random access memory, erasable programmable memory, flash memory, or a combination of one or more types of memory components.

The I/O interface 620 provides communication to and from the various devices within the system 100 and/or system 200 and components of the computing resources to one another. The I/O interface 620 can include one or more input buttons, a communication interface, such as Wi-Fi, Ethernet, or the like, as well as other communication components, such as universal serial bus (USB) cables, or the like.

The power source 625 provides power to the various computing resources and/or devices. The model generation system 100 or shoe scanning and model generation system 200 may include one or more power sources and the types of power source may vary depending on the component receiving power. The power source 625 may include one or more batteries, wall outlet, cable cords (e.g., USB cord), or the like.

The sensors 630 may include sensors incorporated into the model generation system 100 or shoe scanning and model generation system 200 (e.g., image sensors (cameras), light sensors). The sensors 630 are used to provide input to the computing resources that can be used to analyze images, for example.

FIG. 7 is a diagram illustrating a shoe model 720 and a foot model 710 as displayed in a user interface 700. The shoe model 720 can be a shoe model generated using the method according to the flow diagram 500 of FIG. 5 and/or using the system 200 of FIG. 2. The foot model 710 can be a foot model generated using the method according to the flow diagram 300 of FIG. 3 and/or the flow diagram 400 of FIG. 4, and the foot model can be generated using the system 100 of FIG. 1. The display provided via the user interface 700 can comprise individualized shoe information including a composite view of the foot model 710 and the shoe model 720.

As illustrated in the user interface 700, the foot model 710 can be overlaid or superimposed on the shoe model 720, or vice versa. For example, the shoe fit system can place the foot model 710 inside of the shoe model 720 to compare the two models and determine whether the shoe will be a good fit.

In some implementations, the foot model 710 and the shoe model 720 can be displayed to a user in the user interface 700 to allow the user to assess the fit of the shoe. In some examples, volumetric information of the foot model 710, the shoe model 720, or both, may be displayed in addition, or alternatively, or as a part of the foot model, the shoe model, or both. For example, an evaluation of the fit of the shoe can be displayed (e.g., snugness, distance or clearance at one or more points, or the like). In some examples, the foot model 710 is displayed as inside the shoe model 720. In some examples, the shoe model 720 is superimposed or overlaid on the foot model 710. In some examples, the foot model 710, shoe model 720, or both are completely transparent, somewhat transparent, or opaque. The user may be able to investigate one or both models for one or more shoe options to determine a preferred shoe. The user may be able to activate or deactivate the transparency of the shoe model 720 or foot model 710 representation while also spinning or otherwise rotating the combined model or representation.

The user interface 700 can provide various functions for manipulating a view of the foot model 710 and the shoe model 720. For example, a user can click and drag to freely rotate or move the models. A user can zoom in or out on particular portions of the models, such as by selecting a zoom feature 730 (e.g., a plus icon or button to zoom in and a minus icon or button to zoom out or a zoom slider). The user can select a graphic element 740 (e.g., button or icon) to access additional information, such as an assessment generated by the system of the fit of the depicted shoe model (e.g., “good fit,” “too small,” “too big,” “too narrow,” or the like) or other information about the foot model 710 and/or the shoe model 720 (e.g., dimensions, clearance, snugness).

Additionally, the user interface 700 can include a menu 750 comprising a plurality of menu options for configuring the user interface 700. When selected, the menu options included in the menu 750 can configure or reconfigure the user interface 700 in various ways. For example, a “size up” menu option can modify the shoe model 720 to be a shoe model 720 of the next size up, as compared to the currently displayed shoe model 720, and a “size down” menu option can modify the shoe model 720 to be a shoe model 720 of the next size down, as compared to the currently displayed shoe model. A “recommended” menu option can retrieve a shoe model 720 and/or a recommendation of a shoe model 720 generated by the system. For example, the system can generate recommendations of shoe models (e.g., brands or types of shoes) and/or recommended sizes for particular shoe models based on foot models and/or information used to generate foot models, and the “recommended” menu option can cause display of one or more such recommendations. Additionally or alternatively, the “recommended” menu option can cause display of a shoe model 720 for a recommended shoe, and/or the “recommended” menu option can cause display of other information regarding a recommended shoe (e.g., brand, name, size, material information, price, availability).

A “side by side” menu option included in the menu 750 can cause display of multiple foot models 710 and multiple shoe models 720 configured in various ways to facilitate comparison. For example, a side-by-side view generated using the “side by side” menu option can include two foot models 710 and two shoe models 720, each respective foot model 710 and shoe model 720 displayed in separate panes of the user interface 700. A first shoe model 720 may correspond to a first type of shoe (e.g., brand) and a second shoe model 720 may correspond to a second type of shoe. Additionally or alternatively, the first shoe model 720 may correspond to a first size of a type of shoe and a second shoe model may correspond to a second size of the same type of shoe. Any number of panes can be generated in the user interface 700 to facilitate comparisons, and the panes can be configured in various ways (e.g., in any number of rows, columns, grids). In some implementations, a side-by-side view generated using the “side by side” menu option can be generated in an order based on one or more recommendations generated using the system. For example, the system can arrange panes of the side-by-side view such that a first shoe model 720 displayed in the side-by-side view has a highest recommendation score, as compared to additional shoe models 720 displayed in other panes of the side-by-side view. This feature allows a user to more easily identify a best fit and compare a recommended shoe model 720 to one or more alternative shoe models 720.

Additional menu options included in the menu 750 can include an “upload new” menu option, which can allow a user to upload additional data to generate a new foot model 710. For example, upon inspecting the foot model 710 in the user interface 700, a user may determine that the foot model 710 needs to be updated, such as because it appears to be inaccurate. The “upload new” menu option causes display of an interface for generating a new foot model 710, as illustrated in FIGS. 1, 2, 3, and 4. Additionally or alternatively, menu options included in the menu 750 can include a “buy” menu option to place a shoe in a virtual shopping cart and/or initiate a purchase of a shoe corresponding to the shoe model 720. Other menu options may also be provided, and the user interface 700 can be configured or reconfigured in various other ways, such as using controls that are not presented as menu options (e.g., touchscreen features, keys or buttons of a device, voice commands, and the like).

FIG. 8 is a diagram illustrating a shoe model 820 and a foot model 810 as displayed in a user interface 800. The user interface 800 can be as described with reference to the user interface 700 of FIG. 7. In some implementations, the shoe model 820 can be the shoe model 720 of FIG. 7, which is rotated to illustrate a different perspective or point of view, and the foot model 810 can similarly be the foot model 710 of FIG. 7, which is similarly rotated. The user interface 800 can include a zoom feature 830 similar to the zoom feature 730 of FIG. 7, a graphic element 840 similar to the graphic element 740 of FIG. 7, and/or a menu 850 similar to the menu 850 of FIG. 7. The user interface 800 can represent a different perspective or point of view for evaluating a shoe model 820 and a foot model 810. In other words, a user can rotate the models depicted in the user interface 700 of FIG. 7 (e.g., by clicking and dragging) to achieve a different perspective or point of view illustrated in the user interface 800.

Together, the diagrams 700 and 800 allow a user to evaluate the fit of a shoe represented by the shoe model (720/820) as compared to the foot model (710/810) from multiple angles and at multiple points of interest (e.g., at the toe, at the heel, at the arch, at the ankle, etc.). Although particular perspectives are illustrated in the diagrams 700 and 800, the shoe fit system allows for viewing from any perspective, and the perspective can be freely changed, such as by zooming in or out or rotating.

In various examples, the system and method may help to reduce wasted time and money in a user trying to find different shoes that fit his or her feet. Moreover, the disclosed technology overcomes various problems of existing technologies for assessing shoe fit, which are typically based on simplistic measurements, such as length and width, and are poorly suited for choosing shoes without being able to try them on. Additionally, the volumetric comparison (either graphically and/or by presenting numerical information) to a user allows a user to make better informed decisions regarding fit and comfort. Furthermore, in certain instances the system may allow an expansion of ecommerce retail for wearable items, such as shoes and the like. For example, the system may enable users to only be presented with options that are likely to “fit” or feel comfortable to the user based on spacing and fit thresholds and preferences. This helps to enable faster web browsing activities as a website can reduce the number of results and product information that may be presented to a user, allowing faster browsing by the user, and more responsive ecommerce webpages. As a specific example, on a large shoe retailer website, the website may only pull information (e.g., product icons, product page listings, etc.) for shoes that are likely to fit a user, reducing the amount of data needing to be exchanged between the server and user device, as well as speeding up the shopping experience and process by the user. Finally, in instances where the system may compare select volumetric points or locations (rather than a wholesale review), the method executing on a server or user device may be expedited as fewer data points may need to be compared and analyzed, but the fit output may still give an accurate representation to the user as to the likely physical fit of the shoe.

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

1. A method, comprising:
receiving foot data comprising information about a foot of a user;
generating a foot model of the foot based at least in part on the foot data, wherein the foot model comprises a first three-dimensional model representative of a volume of at least a portion of the foot;
comparing the foot model to a shoe model, wherein the shoe model comprises a second three-dimensional model, wherein the shoe model comprises a set of shoe characteristics of a shoe, and wherein the set of shoe characteristics includes at least one material characteristic comprising at least one of a shoe material, a shoe material thickness, an elasticity, a surface characteristic, or a compressability; and
displaying individualized shoe information to the user based at least in part on the comparison, wherein the individualized shoe information includes a fit characteristic based at least in part on the volume and the set of shoe characteristics.

2. The method of claim 1, wherein generating the foot model comprises:
determining the volume of the at least portion of the foot, wherein generating the foot model is based at least in part on determining the volume of the at least portion of the foot.

3. The method of claim 2, wherein the volume is determined at one or more specific locations of the foot.

4. (canceled)

5. The method of claim 2, wherein comparing the foot model to the shoe model comprises:
comparing at least a portion of the volume of the at least portion of the foot to at least a portion of a volume of the shoe, wherein displaying the individualized shoe information to the user is based at least in part on comparing the at least portion of the volume of the at least portion of the foot to the at least portion of the volume of the shoe and wherein the volume of the at least portion of the shoe is based at least in part on the shoe material, the shoe material thickness, a laced-up indication, an environmental indication, or any combination thereof.

6. (canceled)

7. The method of claim 2, further comprising:
comparing a first portion of the volume of the at least portion of the foot to a second portion of the volume of the at least portion of the foot, wherein comparing the foot model to the shoe model is based at least in part on comparing the first portion of the volume of the at least portion of the foot to the second portion of the volume of the at least portion of the foot.

8. The method of claim 1, wherein generating the foot model comprises:
determining a first one-dimensional measurement of a first portion of the at least portion of the foot, wherein generating the foot model is based at least in part on determining the first one-dimensional measurement of the first portion of the at least portion of the foot.

9. The method of claim 8, further comprising:
comparing the first one-dimensional measurement of the first portion of the at least portion of the foot to a second one-dimensional measurement of a second portion of the at least portion of the foot, wherein comparing the foot model to the shoe model is based at least in part on comparing the first one-dimensional measurement of the first portion of the at least portion of the foot to the second one-dimensional measurement of the second portion of the at least portion of the foot.

10. The method of claim 1, wherein generating the foot model comprises:
determining a first area of a first portion of the at least portion of the foot, wherein generating the foot model is based at least in part on determining the first area of the first portion of the at least portion of the foot; and
classifying at least a portion of the at least portion of the foot as a shape based at least in part on receiving the foot data

11. (canceled)

12. (canceled)

13. The method of claim 1, wherein displaying the individualized shoe information to the user comprises:
displaying a superimposed model, wherein the superimposed model comprises the foot model inside the shoe model.

14. (canceled)

15. The method of claim 1, wherein the shoe model further comprises one or more features including one or more one-dimensional shoe measurements, a first volume of the shoe, a second volume of a portion of the shoe, a volume range, a shoe shape for at least a portion of the shoe, a laced-up indication, an environmental indication, or any combination thereof, and wherein comparing the foot model to the shoe model comprises comparing the one or more features of the shoe model to one or more features of the foot model or the at least one material characteristic to the one or more features of the foot model.

16. The method of claim 1, further comprising:
capturing one or more images of the foot, wherein receiving the foot data is based at least in part on capturing the one or more images of the foot.

17. (canceled)

18. (canceled)

19. An apparatus for generating individualized shoe information for a user, comprising:
a camera configured to take one or more pictures of a foot of the user;
a user input interface configured to receive foot data comprising information about the foot;
a processing element configured to:
generate a foot model of the foot based at least in part on the one or more pictures, the foot data, or a combination thereof, wherein the foot model comprises a first three-dimensional model representative of a volume of at least a portion of the foot; and
compare the foot model to a shoe model, wherein the shoe model comprises a second three-dimensional model, wherein the shoe model comprises a set of shoe characteristics of a shoe, and wherein the set of shoe characteristics includes at least one material characteristic comprising at least one of a shoe material, a shoe material thickness, an elasticity, a surface characteristic, or a compressability; and
a display configured to display individualized shoe information to the user based at least in part on the comparison, wherein the individualized shoe information includes a fit characteristic based at least in part on the volume and the set of shoe characteristics.

20. The apparatus of claim 19, wherein generating the foot model comprises:
determining the volume of the at least portion of the foot, wherein generating the foot model is based at least in part on determining the volume of the at least portion of the foot.

21. The apparatus of claim 20, wherein comparing the foot model to the shoe model comprises:
comparing at least a portion of the volume of the at least portion of the foot to at least a portion of a volume of the shoe, wherein displaying the individualized shoe information to the user is based at least in part on comparing the at least portion of the volume of the at least portion of the foot to the at least portion of the volume of the shoe.

22. (canceled)

23. (canceled)

24. (canceled)

25. The apparatus of claim 24, wherein the processing element is further configured to:
compare the first one-dimensional measurement of the first portion of the at least portion of the foot to a second one-dimensional measurement of a second portion of the at least portion of the foot, wherein comparing the foot model to the shoe model is based at least in part on comparing the first one-dimensional measurement of the first portion of the at least portion of the foot to the second one-dimensional measurement of the second portion of the at least portion of the foot.

26. The apparatus of claim 19, wherein generating the foot model comprises:
determining a first area of a first portion of the at least portion of the foot, wherein generating the foot model is based at least in part on determining the first area of the first portion of the at least portion of the foot; and
compare the first area of the first portion of the at least portion of the foot to a second area of the second portion of the at least portion of the foot, wherein comparing the foot model to the shoe model is based at least in part on comparing the first area of the first portion of the at least portion of the foot to the second area of the second portion of the at least portion of the foot.

27. (canceled)

28. The apparatus of claim 19, wherein generating the foot model further comprises:
classifying at least a portion of the at least portion of the foot as a shape based at least in part on receiving the foot data.

29. The apparatus of claim 19, wherein displaying the individualized shoe information to the user comprises:
displaying a superimposed model, wherein the superimposed model comprises the foot model inside the shoe model; and
displaying one or more shoe options to the user.

30. (canceled)

31. (canceled)

32. (canceled)

33. The apparatus of claim 19, wherein at least one of the one or more pictures includes a reference item, and wherein generating the foot model is based at least in part on the at least one of the one or more pictures including the reference item.

34. The apparatus of claim 19, wherein the processing element is further configured to:
provide a user interface via the display, wherein the user interface is configured to display the individualized shoe information, and wherein the individualized shoe information includes a composite view of the foot model and the shoe model;
receive a selection, via the user interface, of a command to change a view in the user interface, wherein the change of the view comprises at least one of zooming the view, rotating the view, or modifying the shoe model; and
change the view responsive to the selection.
